(19) United States
(12) Patent Application Publication
(10) Pub. No.: US 2015/0216739 A1
(43) Pub. Date: Aug. 6, 2015

(54) DISPOSABLE BIODEGRADABLE DIAPERS

(71) Applicants: Tom Datwyler, Hudson, WI (US); John D. McCarthy, Hudson, WI (US)

(72) Inventors: Tom Datwyler, Hudson, WI (US); John D. McCarthy, Hudson, WI (US)

(21) Appl. No.: 14/615,081

(22) Filed: Feb. 5, 2015

Related U.S. Application Data
(60) Provisional application No. 61/935,881, filed on Feb. 5, 2014.

(51) Int. Cl. A61F 13/505

(52) U.S. Cl. CPC A61F 13/505

(57) ABSTRACT

A diaper system for infants including a disposable, biodegradable inner insert and a reusable, washable outer cover. The insert is disposed next to the baby's body and absorbs liquid waste and captures solid waste. The cover holds the insert securely in place on the infant and serves as a backup for retention of liquid and solid waste. Periodically, the system is changed whereby the cover is removed, the insert is then removed from the infant and disposed of in garbage or the like, where it later biodegrades. The baby is cleaned and a fresh insert is applied. The same or a new cover is then applied and secured to hold the insert in place. The process can be repeated indefinitely and a relatively small number of reusable covers (for example 2 or 3) can be used, and cleaned periodically, with a larger number of disposable and biodegradable inserts (for example approximately 100 per month). Embodiments of the disposable insert and reusable cover are also disclosed. Finally, a one-piece diaper is disclosed that may optionally be used with a reusable cover.

### 37 C.F.R. §1.71(e) AUTHORIZATION

A portion of the disclosure of this patent document contains material which is subject to copyright protection. The copyright owner has no objection to the facsimile reproduction by anyone of the patent document or the patent disclosure, as it appears in the US Patent and Trademark Office patent file or records, but otherwise reserves all copyright rights whatsoever.

### CROSS-REFERENCE TO RELATED APPLICATIONS, IF ANY

This application claims the benefit under 35 U.S.C. §119(e) of co-pending U.S. Provisional Patent Application Ser. No. 61/935,881, filed Feb. 5, 2014, which is hereby incorporated by reference.

### STATEMENT REGARDING FEDERALLY SPONSORED RESEARCH OR DEVELOPMENT

Not applicable.

### REFERENCE TO A MICROFICHE APPENDIX, IF ANY

Not applicable.

### BACKGROUND OF THE INVENTION

1. Field of the Invention

The present invention relates, generally, to infant diaper systems, apparatus and methods. Particularly, the invention relates to a diaper system having a disposable, biodegradable insert and a reusable, washable cover, and to a diaper that is completely disposable.

2. Background Information.

Existing technology in this field is believed to have significant limitations and shortcomings.

All US patents and patent applications, and all other published documents mentioned anywhere in this application are incorporated by reference in their entirety.

### BRIEF SUMMARY OF THE INVENTION

The present invention provides diapers and diaper methods which are practical, reliable, accurate and efficient, and which are believed to constitute an improvement over the background technology.

In one aspect, the invention provides a diaper system for infants including a disposable, biodegradable inner insert and a reusable, washable outer cover. The insert is disposed next to the baby's body and absorbs liquid waste and captures solid waste. The cover holds the insert securely in place on the infant and serves as a backup for retention of liquid and solid waste. Periodically, the system is changed whereby the cover is removed, the insert is then removed from the infant and disposed of in garbage or the like, where it later biodegrades. The baby is cleaned and a fresh insert is applied. The same or a new cover is then applied and secured to hold the insert in place. The process can be repeated indefinitely and a relatively small number of reusable covers (for example 2 or 3) can be used, and cleaned periodically, with a larger number of disposable and biodegradable inserts (for example approximately 100 per month).

In another embodiment, the invention provides a diaper that is completely disposable. The disposable diaper may be further used with a washable, reusable outer cover for extra protection.

The present invention is believed to involve novel elements, combined in novel ways to yield more than predictable results. The problems solved by the invention were not fully recognized in the prior art.

The aspects, features, advantages, benefits and objects of the invention will become clear to those skilled in the art by reference to the following description, claims and drawings.

### BRIEF DESCRIPTION OF THE SEVERAL VIEWS OF THE DRAWING

FIG. 1 is a perspective view of an embodiment of the diaper system of the present invention including a disposable, biodegradable insert and a reusable cover, shown in a condition for placement on an infant.

FIG. 2 is a perspective view of the diaper system of FIG. 1, shown as it would appear deployed on an infant.

FIG. 3 is a top or interior view of the diaper system, shown substantially flat or open.

FIG. 4 is a bottom or exterior view of the diaper system, also shown flat.

FIG. 5 is an end view of the diaper system.

FIG. 6 is an opposite end view of the diaper system.

FIG. 7A crossectional view of the diaper system taken along line 7-7 of FIG. 3, this embodiment of the system having a cover with two body layers.

FIG. 8A is a detailed view of the area -8A- of FIG. 7A.

FIG. 9A is a detailed view of area -9A- of FIG. 7A.

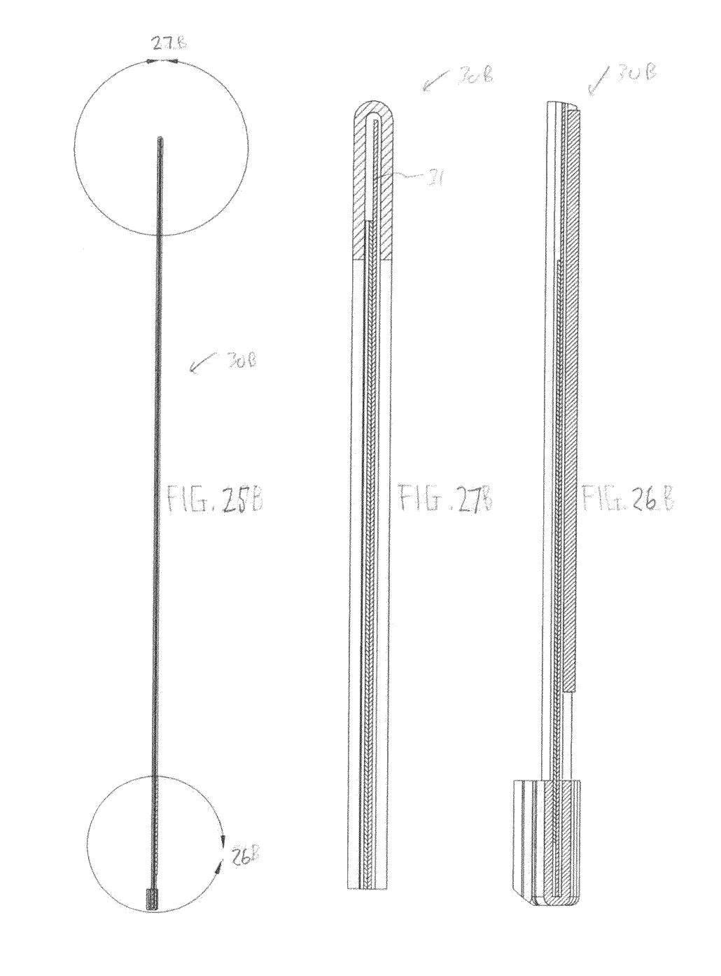

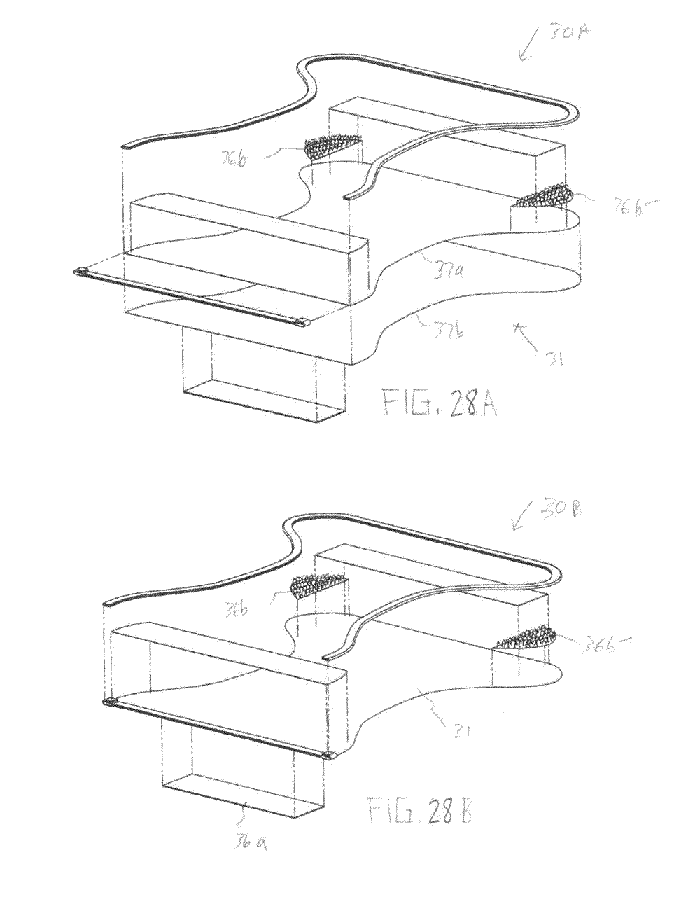

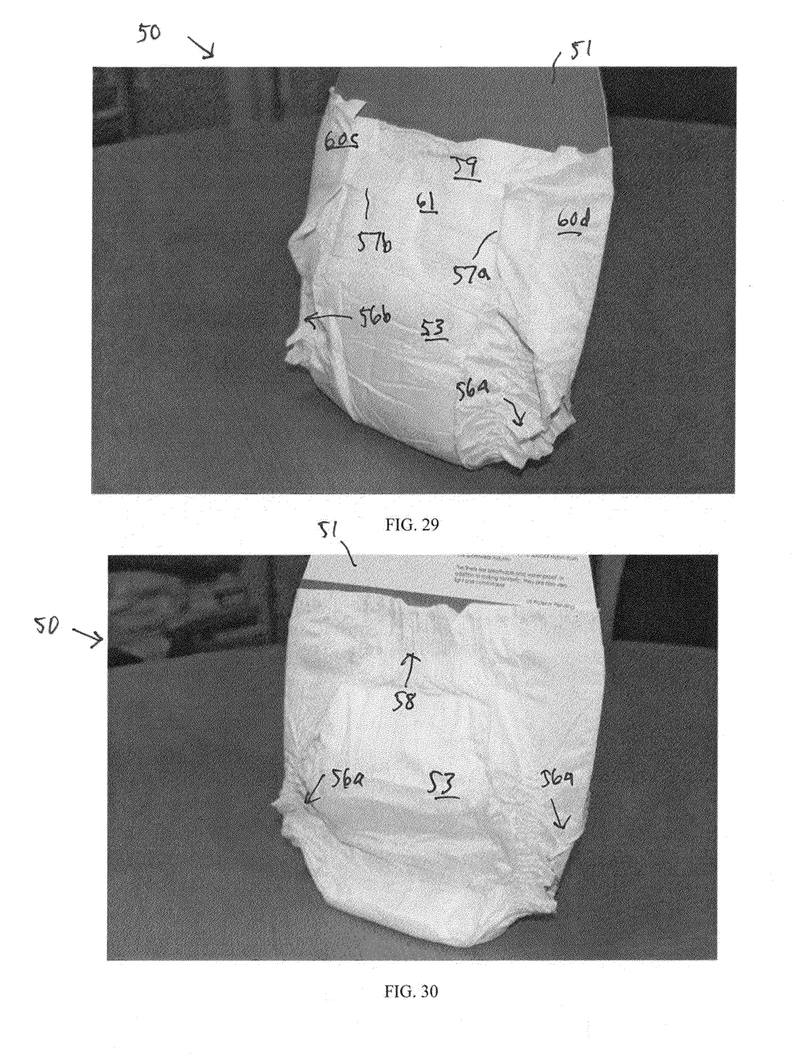

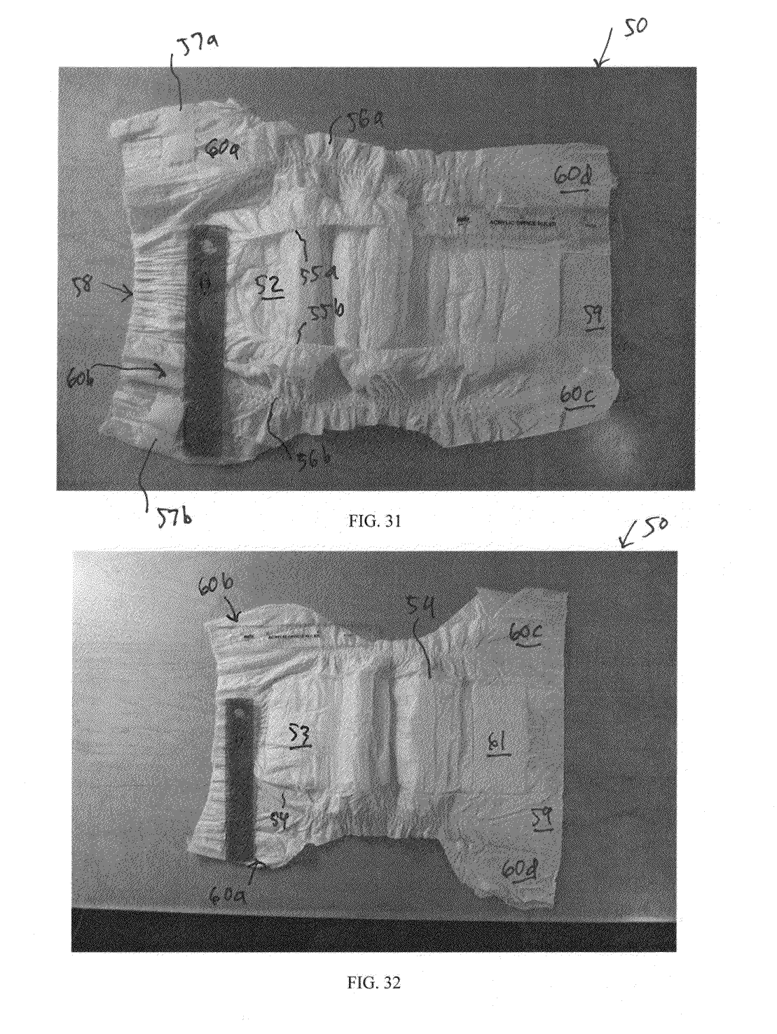

FIGS. 7B, 8B and 9B are crossectional views of an alternative embodiment of the diaper system wherein the cover has a single layer of outer fabric, as opposed to two layers as in FIGS. 7A, 8A, and 9A.

FIG. 10 is an exploded view of the diaper system of the invention.

FIG. 11 is a perspective view of an embodiment of the disposable, biodegradable diaper insert of the present invention for use with a reusable cover in a diaper system, shown flat and in a condition for placement in a cover and on an infant.

FIG. 12 is a perspective view of the diaper insert of FIG. 11, shown substantially as it would appear deployed on an infant inside the cover of the system.

FIG. 13 is a top or interior view of the insert, shown substantially flat or open.

FIG. 14 is a bottom or exterior view of the insert, also shown flat.

FIG. 15 is a side view of the insert.

FIG. 16 crossectional view of the insert taken along line 16-16 of FIG. 14.

FIG. 17 is a detailed view of the portion identified as -17- in FIG. 16.

FIG. 18 is a perspective view of an embodiment of a reusable cover of the present invention for use with a disposable, biodegradable diaper insert, shown in a condition for placement on an infant.

FIG. 19 is a perspective view of the reusable cover of FIG. 18, shown as it would appear deployed on an infant.

FIG. 20 is a top or interior view of the cover, shown flat or open.

FIG. 21 is a bottom or exterior view of the cover, also shown flat.

FIG. 22 is an end view of the cover.

FIG. 23 is an opposite end view of the cover.

FIG. 24 is a side view of the cover.

FIG. 25A crossectional view of the cover taken along line 25-25 of FIG. 21, this embodiment of the cover having two body layers.

FIG. 26A is a detailed view of the area -26A- of FIG. 25A.

FIG. 27A is a detailed view of the area -27A- of FIG. 25A.

FIG. 25B crossectional view of the cover taken along line 25-25 of FIG. 21, this embodiment of the cover having a single body layer.

FIG. 26B is a detailed view of the area -26B- of FIG. 25B.

FIG. 27B is a detailed view of the area -27B- of FIG. 25B.

FIGS. 28A and 28B are exploded views of the cover embodiments having two and one layers respectively.

FIG. 29 is a front perspective view of an alternative embodiment of the diaper of the present invention that consists of a disposable, biodegradable diaper that is completely disposable and does not require a separate cover, in a condition as it would be placed on an infant, and an insert exposed for clarity.

FIG. 30 shows back perspective view of the diaper of FIG. 29.

FIG. 31 a top view of the diaper interior side, open and laid out substantially flat.

FIG. 32 is a top view of the diaper exterior side, laid out substantially flat.

### DETAILED DESCRIPTION

Diapers are a major source of buildup in landfills because existing disposable diapers are either completely non-biodegradable, or have a large number of non-biodegradable component. The present invention solves this problem by maximizing the number of biodegradable components and thus minimizing waste accumulation.

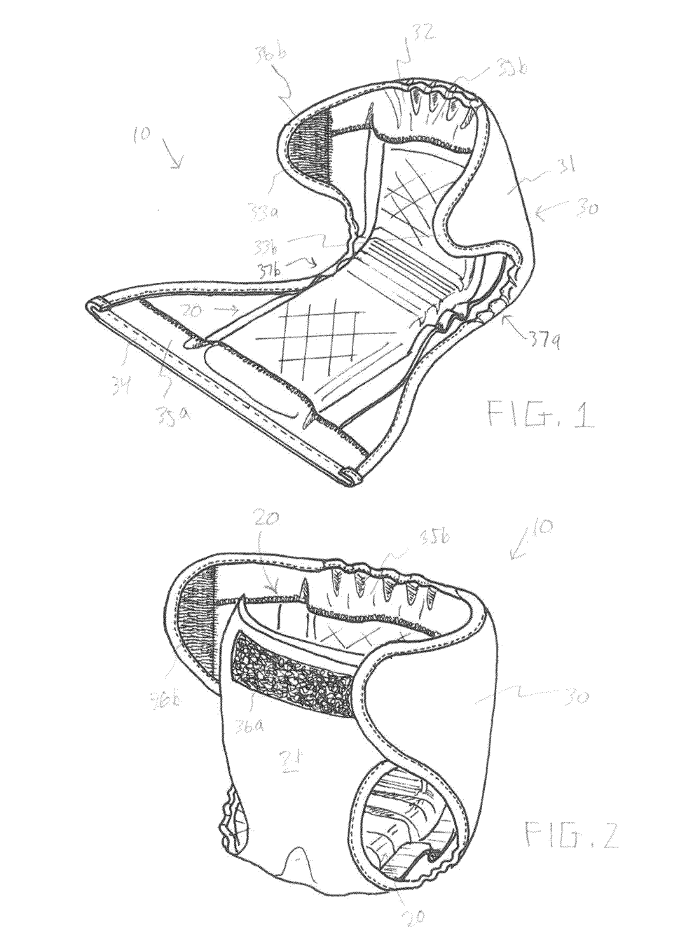

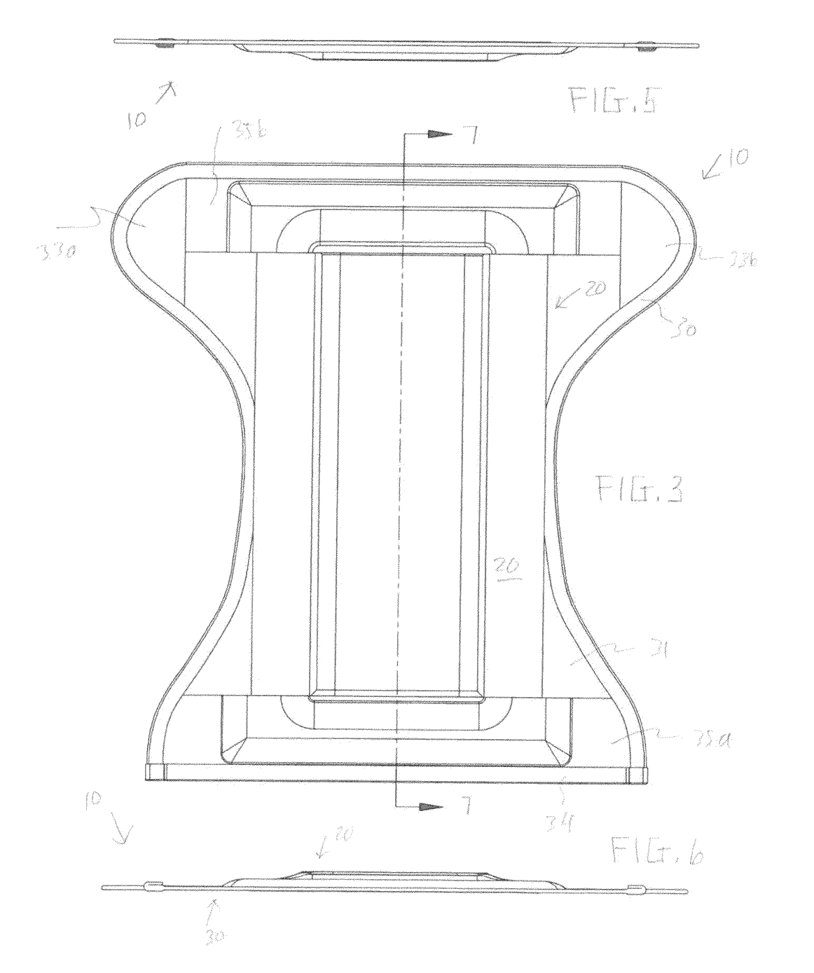

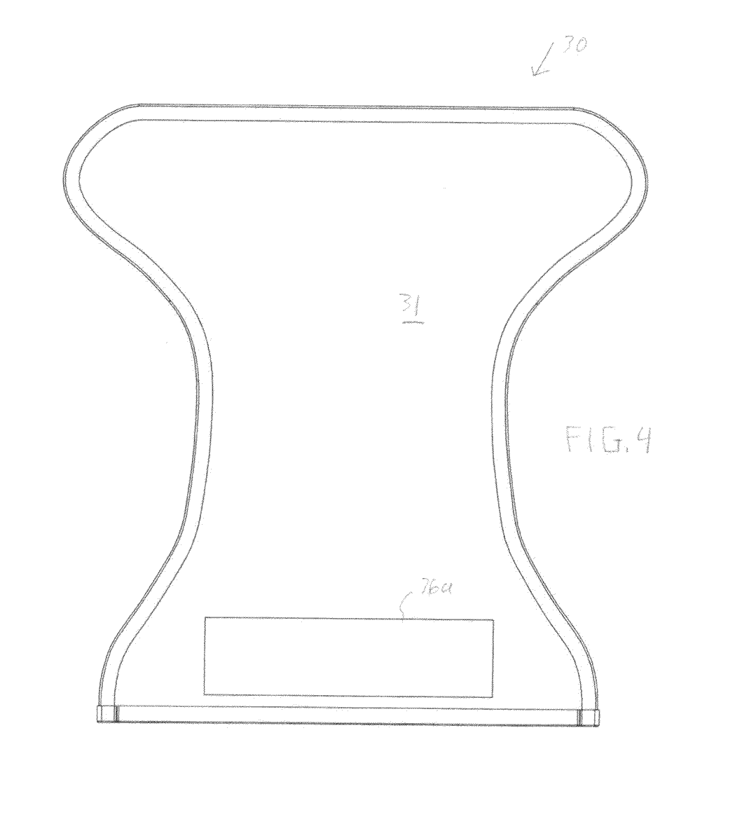

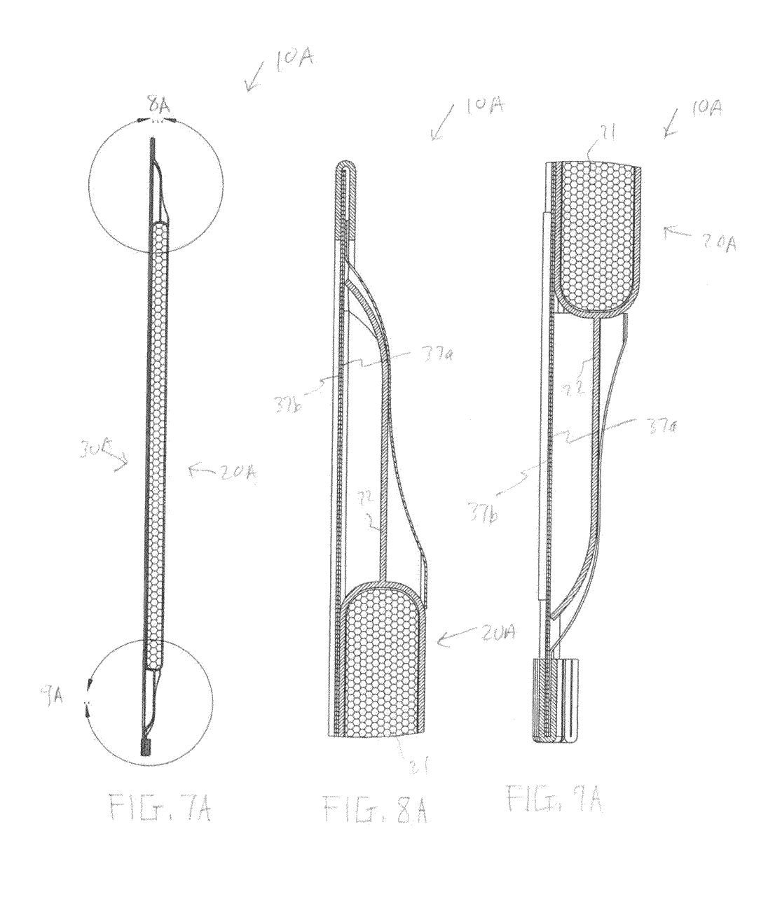

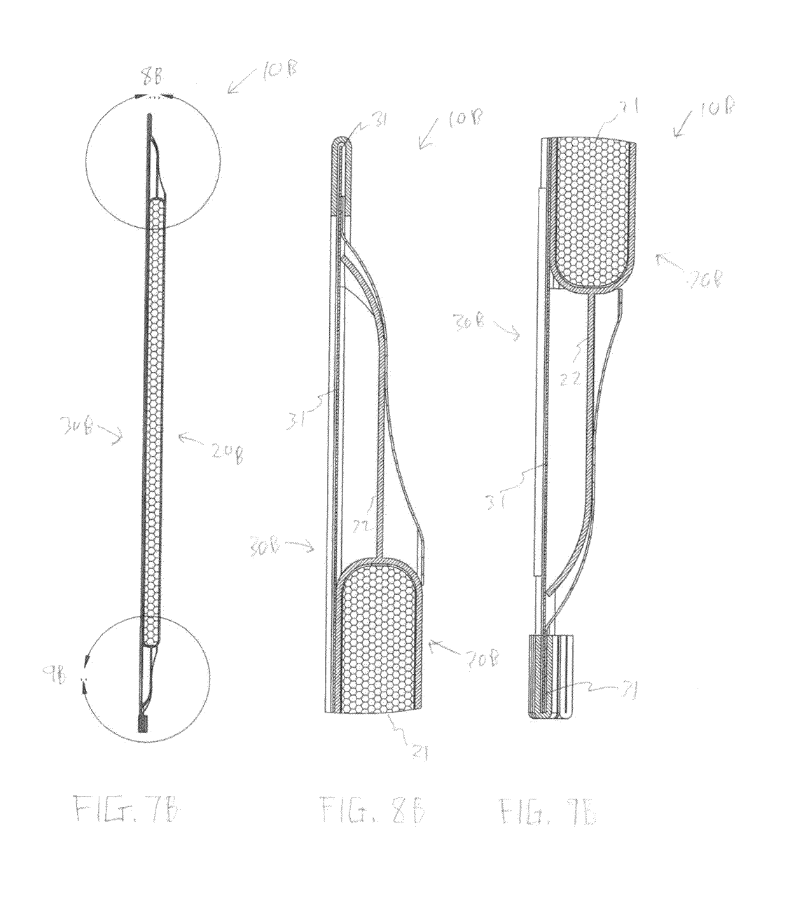

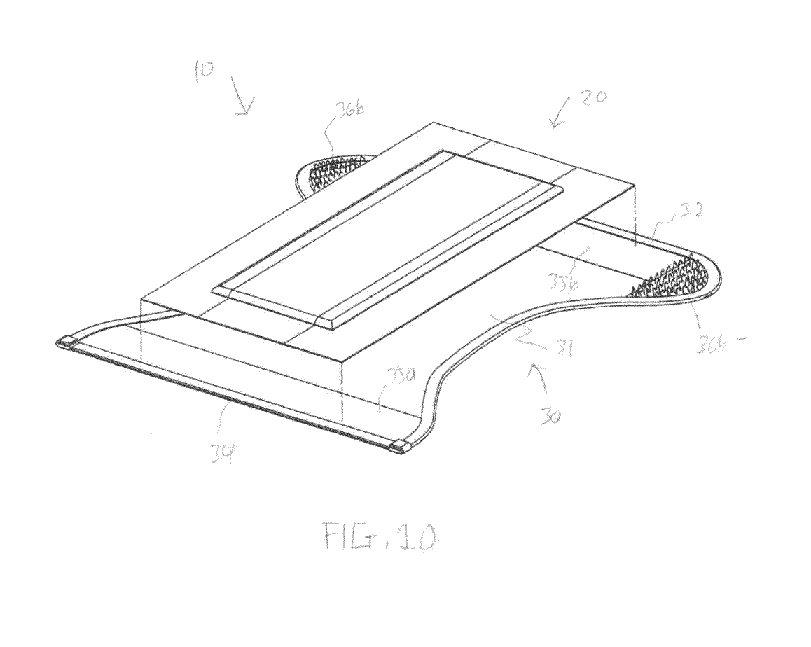

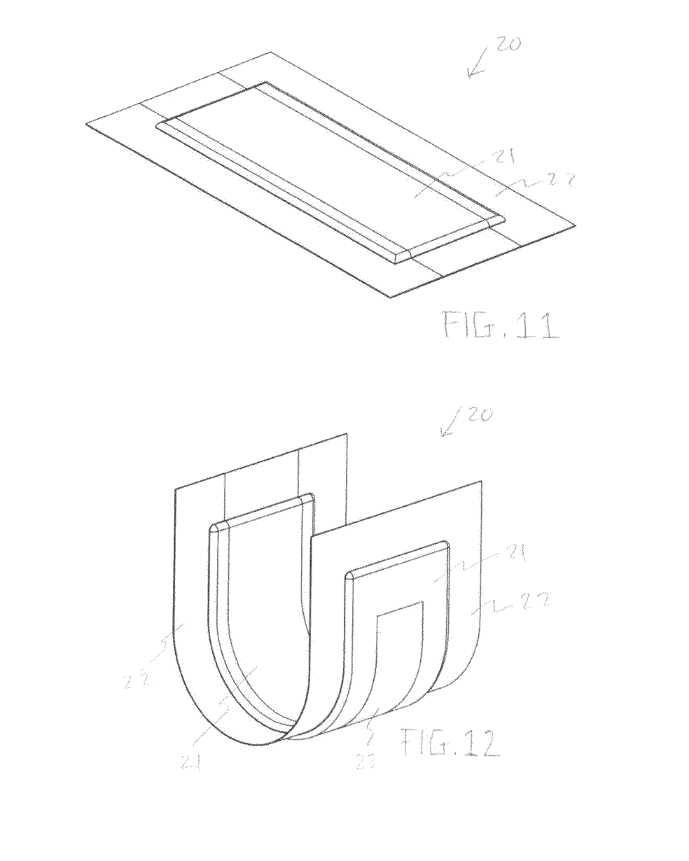

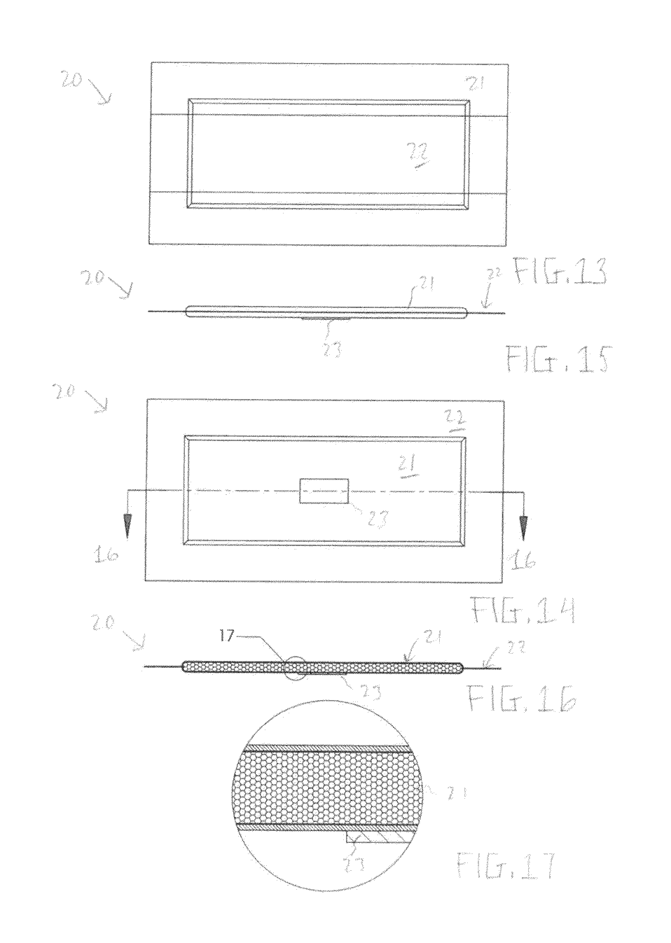

Referring to FIGS. 1-10, an embodiment of the diaper system 10 for infants includes a disposable, biodegradable inner insert 20 and a reusable, washable outer cover 30. The insert 20 is disposed next to the baby's body and absorbs liquid waste and captures solid waste. The cover 30 holds the insert 20 securely in place on the infant and serves as a backup for retention of liquid and solid waste. Periodically, the system is changed whereby the cover 30 is removed, the insert 20 is then removed from the infant and disposed of in garbage or the like, where it later biodegrades. The baby is cleaned and a fresh insert 20 is applied. The same or a new cover 30 is then applied and secured to hold the fresh insert 20 in place. The process can be repeated indefinitely and a relatively small number of reusable covers 30 (for example 2 or 3) can be used, and cleaned periodically, with a larger number of disposable and biodegradable inserts 20 (for example approximately 100 per month).

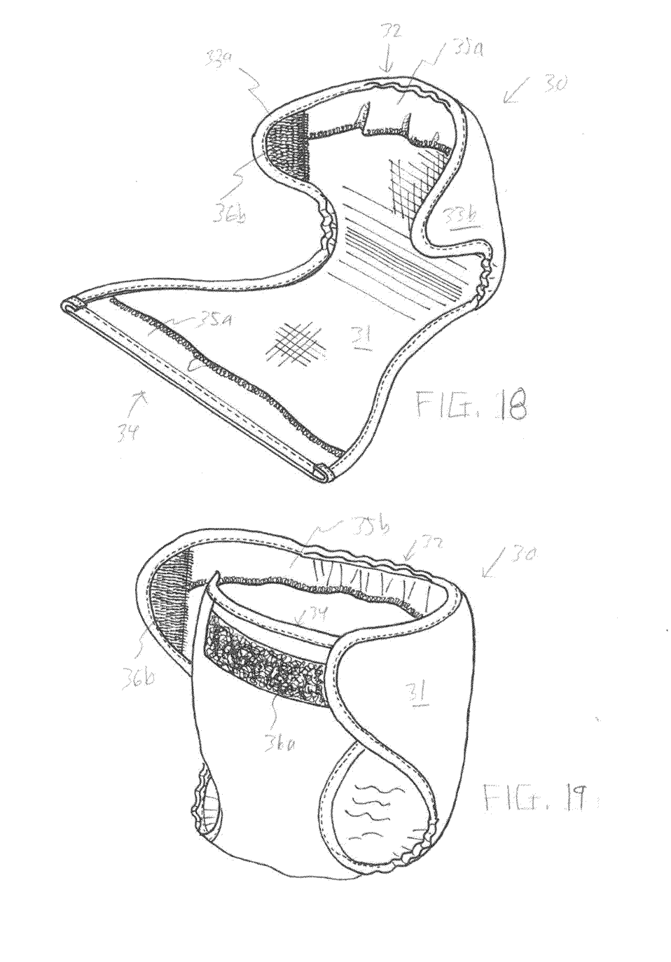

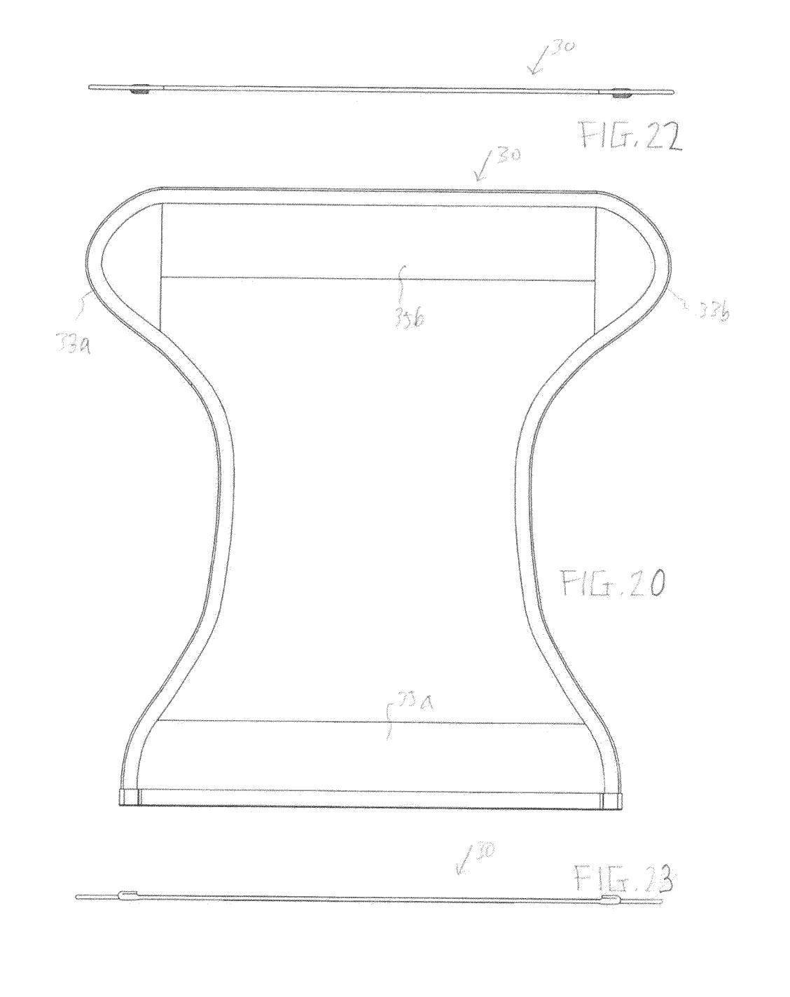

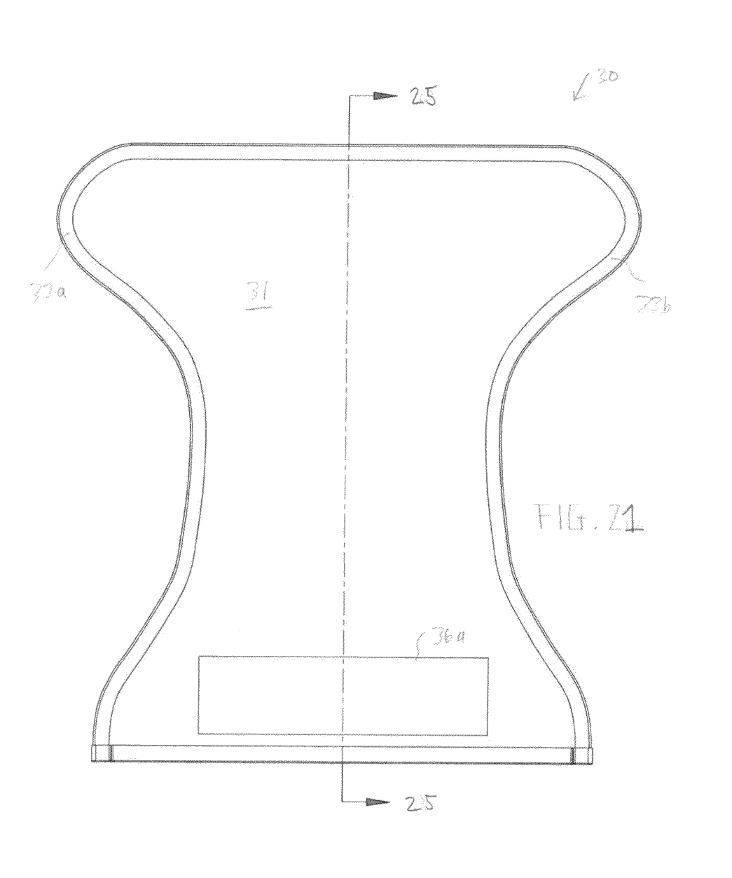

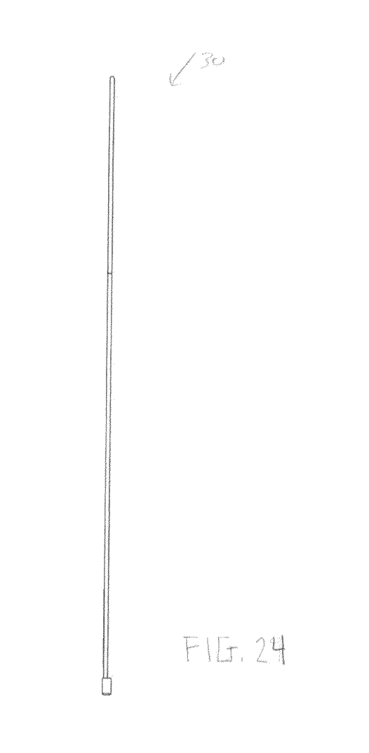

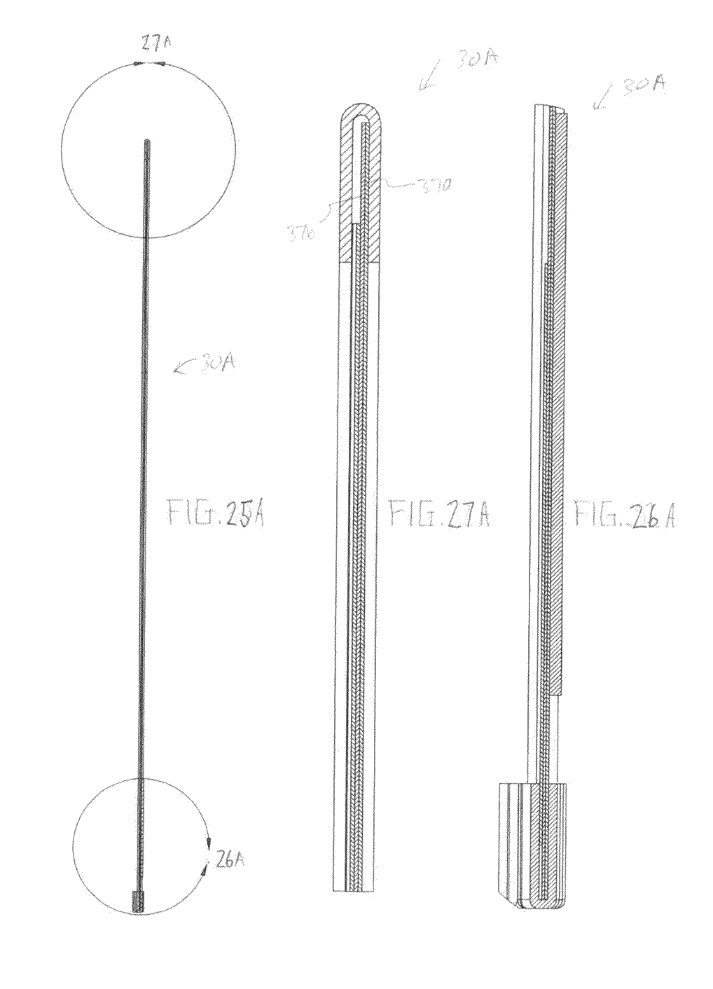

Referring to FIGS. 1 and 2, the reusable, washable cover 30 preferably has a curved, butterfly-type configuration adapted to mate with the mid-section of a human infant. The cover 30 has a base or body member 31, with a top back waist band 32, a pair of flaps 33a,b, and a top front waist band 34. Elastic leg cuffs 37a/b are preferably disposed peripherally at the mid-portion of the insert. On the inside, a pair of front and back insert cuff or awnings 35a/b engage and hold the ends of the insert 20. The top front waste band 34 has a hook and loop type first member 36a, and the back flaps 33a/b have the complementary second member 36b.

Referring also to FIGS. 18-24, the cover 30 is preferably constructed of flexible material such as fabric or polymeric materials. The cover 30 may be constructed of durable, non-biodegradable materials. The cover is preferably constructed of a breathable, waterproof material, such as Nylon. The cover also preferably has hook and loop fastener panels for securement around an infant. The cover 30 is preferably machine or hand washable. The outer surface of the cover 30 may be constructed of a moisture resistant material or coated with a moisture resistant coating. In the cover 30A embodiment of FIGS. 7A, 8A, 9A and 30A to 33A, the cover 30 body 31 has a pair of layers 37, one interior 37a and one exterior 37b. Alternatively, in the cover 30B embodiment shown in FIGS. 7B, 8B, 9B and 30B to 33B, the body 31 has a single layer 31.

Referring to FIGS. 11-17, a preferred embodiment of the insert 20 is constructed completely or substantially completely of biodegradable materials so that when it is removed from the infant and diaper system 10 and disposed of it will completely degrade and not contribute to long term waste accumulation in the environment. The outwardly facing surface of the insert 20 may be constructed of a moisture resistant material or coated with a moisture resistant substance. In this embodiment, the insert 20 has a generally flat, rectangular configuration with a central absorbent region 21 and a peripheral region 22. The insert preferably has a central adhesive strip 23 on its outer or exterior surface for releasable securement with the cover 30. Alternatively, the insert may have a curved or body shaped configuration (for example similar or identical to that of the article shown in FIGS. 29-32), again with a central absorbent region and outer region. The insert may also having intermediary flap and tapered leg regions. Finally, the insert may have an outer shell layer made for example of PLA Bio-Plastic. The filling material consists of wood pulp. The inner layer is preferably bamboo material.

The cover/insert system 10 may be packaged in a set of 1 to 3 covers 30 along with 10 to 30 inserts 20. Additional inserts may be purchased in bulk. The package may be constructed in such a way as to serve as a receptacle for soiled inserts 20. The package itself may be constructed of biodegradable material.

FIGS. 29-32 show a further alternative embodiment of the diaper system 50 of the invention, attached to a paperboard package element 51. This embodiment only has one diaper element 50 or piece. Unlike prior embodiments, no separate cover is required. The diaper 50 is constructed of an inside contact layer 52, an outer base layer 53, and an absorbent core (the outline 54 of which is best shown in FIG. 32) disposed between the inner and outer layers 52 and 53. All of these layers 52, 53 and 54 are shown to have a rectangular configuration. Inner, elastic leak guards 55a/b and outer leg cuffs 56a/b are disposed peripherally at the mid-portion of the insert. A pair of adhesive tabs 57a/b are disposed near the top diaper for securing the diaper to an infant. The back, upper edge of the diaper 50 also preferably has an elastic band 58 for securement at the waist of the infant. The front upper edge 59 is preferably flat and is substantially inelastic. However, the wing shaped corners 60 a, b, c, and d of the diaper 50 are preferably elastic for stretching contact with the infant. A tape contact element 61 is preferably disposed on the upper front exterior surface 53 for engagement by the adhesive tabs 57 a/b.

Significantly, both the inside and outside layers are constructed of bamboo material. The interior fill is constructed of wood pulp. There is no polymeric material used in the any of these layers. This permits substantially complete biodegradation in as little as two (2) months. In contrast, common biodegradable inserts degrade in approximately two years. The diaper of the invention therefore fully complies with EN 13432 Europe and ASTM-D6400 US standards for compostability.

FIGS. 29-30 show the outside of the diaper, in an operative orientation. FIG. 31 shows the inside of the diaper, laid substantially flat and open. Side tapes mare made from a non-biodegradable material, but are designed to easily tear away after use for removal prior to composting. FIG. 32 shows the outside of the diaper when flat. The outside may also have a corn starch based waterproof biofilm or layer. This embodiment of the diaper may be covered by an optional cover, if desired by the user.

The embodiments above are chosen, described and illustrated so that persons skilled in the art will be able to understand the invention and the manner and process of making and using it. The descriptions and the accompanying drawings should be interpreted in the illustrative and not the exhaustive or limited sense. The invention is not intended to be limited to the exact forms disclosed. While the application attempts to disclose all of the embodiments of the invention that are reasonably foreseeable, there may be unforeseeable insubstantial modifications that remain as equivalents. It should be understood by persons skilled in the art that there may be other embodiments than those disclosed which fall within the scope of the invention as defined by the claims. Where a claim, if any, is expressed as a means or step for performing a specified function it is intended that such claim be construed to cover the corresponding structure, material, or acts described in the specification and equivalents thereof, including both structural equivalents and equivalent structures, material-based equivalents and equivalent materials, and act-based equivalents and equivalent acts.

The invention claimed is:

1. A diaper system comprising a disposable insert and a reusable cover, the disposable insert being adapted to be coupled to the cover, the insert being adapted to be placed against the skin of a person, the cover being adapted to secure around the body of a person.

2. The diaper system of claim 1, wherein the cover is constructed of flexible fabric and has an interior side, the insert being coupled to the interior side of the cover.

3. The diaper system of claim 2, has at least one cuff disposed on the interior side of the cover for engaging the insert, whereby in use, a portion of the insert is placed in the cuff.

4. The diaper system of claim 3, wherein there are two cuffs, one cuff disposed along a bottom edge of the cover and the second cuff being disposed along the top edge of the cover.

5. The diaper system of claim 4, wherein the cover has at least two flaps disposed at the top region of the cover, the flaps being adapted to couple with the bottom region of the cover when the cover is placed on the person.

6. The diaper of claim 5, wherein the cover has a first hook and loop type connection member disposed at the bottom region, on the exterior side thereof, and wherein the cover has complementary second hook and loop type connection members disposed at each flap, on the interior side thereof.

7. The diaper of claim 6, wherein the cover has elastic leg cuff members peripherally at a mid region thereof.

8. The diaper of claim 1, wherein the insert is constructed completely of biodegradable materials.

9. The diaper of claim 8, wherein the insert has a substantially rectangular and has an inner layer adapted to be placed against the skin of the user, an outer layer adapted to abut the cover, and an absorbent layer disposed between the inner and outer layer.

10. The diaper of claim 9, wherein the inner and outer layer are constructed of bamboo material and the absorbent layer is constructed of wood pulp material.

11. A disposable, biodegradable diaper adapted to secure around the body of a person, the diaper comprising a inner layer adapted to be placed against the skin of the user, an outer layer adapted to abut the cover, and an absorbent layer disposed between the inner and outer layer, wherein the inner and outer layer are constructed of bamboo material and the absorbent layer is constructed of a biodegradable material, the diaper further comprising means to attach the diaper to a person.

12. The diaper of claim 11, wherein the absorbent layer biodegradable material includes wood pulp.

13. The diaper of claim 11, wherein the inner layer, outer layer and absorbent layer have a substantially coextensive rectangular configuration.

14. The diaper of claim 13, further comprising a wing regions disposed at each corner.

15. The diaper of claim 14, wherein the wing regions are stretchable.

16. The diaper of claim 14, further comprising an elastomeric band at a top end thereof.

17. The diaper of claim 14, further comprising an elastic leak guard disposed peripherally at the midportion thereof.

18. The diaper of claim 17, further comprising an outer leg cuff disposed peripherally at the midportion thereof.

19. The diaper of claim 1, wherein the attachment means comprises at least two adhesive members.

20. A diaper system for use with a human being, comprising:
a. a disposable insert, the insert including:
i. an inner layer adapted to be placed against the skin of the user, an outer layer adapted to abut the cover, and an absorbent layer disposed between the inner and outer layer, wherein the inner and outer layer are constructed of bamboo material and the absorbent layer is constructed of a biodegradable material, the diaper further comprising means to attach the diaper to a person, and
ii. wherein the inner layer, outer layer and absorbent layer have a substantially coextensive rectangular configuration; and
b. a reusable cover, the disposable insert being adapted to be coupled to the cover, the insert being adapted to be placed against the skin of a person, the cover being adapted to secure around the body of a person, the cover:
i. being constructed of flexible fabric and has an interior side, the insert being coupled to the interior side of the cover,
ii. having two cuffs disposed on the interior side of the cover for engaging the insert, whereby in use, a portion of the insert is placed in each cuff, one cuff disposed along a bottom edge of the cover and the second cuff being disposed along the top edge of the cover,
iii. having at least two flaps disposed at the top region of the cover, the flaps being adapted to couple with the bottom region of the cover when the cover is placed on the person, and,
iv. having a first hook and loop type connection member disposed at the bottom region, on the exterior side thereof, and wherein the cover has complementary second hook and loop type connection members disposed at each flap, on the interior side thereof.
